(19) United States
(12) Patent Application Publication
(10) Pub. No.: US 2013/0309713 A1
(43) Pub. Date: Nov. 21, 2013

(54) PATHOGEN DETECTION SYSTEMS AND METHODS

(71) Applicants: David Ribble, Indianapolis, IN (US); Michelle McCleerey, Raleigh, NC (US); Eric Agdeppa, Batesville, IN (US); Brian Lawrence, Cincinnati, OH (US); Jason Penninger, Indianapolis, IN (US)

(72) Inventors: David Ribble, Indianapolis, IN (US); Michelle McCleerey, Raleigh, NC (US); Eric Agdeppa, Batesville, IN (US); Brian Lawrence, Cincinnati, OH (US); Jason Penninger, Indianapolis, IN (US)

(21) Appl. No.: 13/654,649

(22) Filed: Oct. 18, 2012

Related U.S. Application Data
(60) Provisional application No. 61/647,753, filed on May 16, 2012.

(51) Int. Cl. C12M 1/34; G01N 33/00; C12Q 1/04

(52) U.S. Cl. 435/34; 435/287.1; 73/23.34

(57) ABSTRACT

Pathogen and chemical detection systems for use with a patient support apparatus and related methods are disclosed. A fluid or biologic sample from the vicinity of a person supported by a person support apparatus is acquired and transmitted to a pathogen detection sensor. A graphical caregiver interface is supplied output from the pathogen detection sensor and detects the presence of a chemical. The graphical caregiver interface communicates with an electronic medical records database, and alarm and a nurse call system.

### CROSS-REFERENCE TO RELATED PATENT APPLICATIONS

This patent application claims the benefit of U.S. Provisional Patent Application 61/647,753 filed on May 16, 2012, the entire disclosure of which is hereby incorporated by reference.

### BACKGROUND

Detection of pathogens and odors in the vicinity of a patient supported by a person support apparatus is a continuing challenge. Secretions of chemicals and/or biomarkers by a patient may be indicative of presence of pathogens, the rate of change in development of certain conditions and/or onset of other conditions. While several systems exist to detect and monitor pathogens a need exists to continue development in this area.

### BRIEF SUMMARY

The present disclosure includes one or more of the features recited in the appended claims and/or the following features which, alone or in any combination, may comprise patentable subject matter.

One embodiment of a system for detecting pathogens for use with a person support apparatus comprises a fluid transport system configured to transport fluid away from skin of a person supported by a person support apparatus. At least one pathogen detection sensor is configured to detect a pathogen in fluid transported in the fluid transport system. A caregiver interface, the pathogen detection sensor is configured to communicate with the caregiver interface which is configured to determine presence of a pathogen in a quantity exceeding a predetermined threshold, the caregiver interface configured to control at least one function of a person support apparatus.

Another embodiment of a system for detecting an odor in the vicinity of a hospital bed comprises at least one odor sensor to which a fluid transport system transports fluid from skin of a person supported by a hospital bed. The at least one odor sensor configured to communicate with a caregiver interface.

An embodiment of system for purification of air in the vicinity of a person supported by a person support apparatus comprises a person support apparatus and a fluid transport system configured to transport fluid away from a person supported by the person support apparatus. At least one chemical sensor is configured to sense at least one chemical in fluid transported away from a person supported by the person support apparatus. The chemical sensor is configured to communicate with a caregiver interface and the caregiver interface is also configured to communicate with an air filtration system.

One embodiment system for monitoring wound development comprises a person support apparatus and a fluid transport system configured to transport fluid away from a person supported by the person support apparatus. At least one chemical sensor is configured to sense at least one chemical in fluid transported away from a person supported by the person support apparatus. A caregiver interface, the chemical sensor is configured to communicate with the caregiver interface, the caregiver interface comprising a memory configured to store signals supplied by the chemical sensor for a predetermined time.

One embodiment of a method for detecting a chemical in the vicinity of a person support apparatus comprises the operation of transporting fluid away from a person supported by a person support apparatus, testing the fluid transported from a person supported by the person support apparatus and reporting result of said testing to a caregiver interface. The method comprises a further operation of determining whether said result indicates presence of at least one chemical by determining whether quantity of the at least one chemical detected is greater than a predetermined threshold. An alarm is activated to alert a caregiver when quantity of the at least one chemical detected is greater than predetermined threshold.

### BRIEF DESCRIPTION OF DRAWINGS

The accompanying drawings incorporated in and forming a part of the specification illustrate several aspects of the claimed subject matter and, together with the description, serve to explain the principles of the claimed subject matter. In the drawings:

FIG. 1 is a block diagram of a system for detecting pathogens for use with a person support apparatus wherein a portion of the fluid transport system comprises a mattress topper, constructed according to one or more of the principles disclosed herein;

FIG. 2 is a block diagram of another system for detecting pathogens for use with a person support apparatus wherein at least a portion of the pathogen sensor is housed within a mattress topper, constructed according to one or more of the principles disclosed herein;

FIG. 3 is a block diagram of another system for detecting pathogens for use with a person support apparatus wherein a pathogen containment screen is configured to envelope at least a portion of a patient supported by a person support apparatus, constructed according to one or more of the principles disclosed herein;

FIG. 4 is a block diagram of another system for detecting pathogens for use with a person support apparatus wherein at electric potential is used to generate fluid flow in a fluid transport system, constructed according to one or more of the principles disclosed herein;

FIG. 5 is a block diagram of a system for detecting pathogens for use with a person support apparatus wherein the pathogen sensing system is a free standing device, constructed according to one or more of the principles disclosed herein;

FIG. 6 is a block diagram of another system for detecting pathogens for use with a person support apparatus wherein the pathogen sensing system is housed within the air filtration system, constructed according to one or more of the principles disclosed herein;

FIG. 7 is a block diagram of another system for detecting pathogens for use with a person support apparatus wherein the caregiver interface comprises a controller, a display device and a caregiver input device, constructed according to one or more of the principles disclosed herein

### DETAILED DESCRIPTION OF ILLUSTRATIVE EMBODIMENTS

The embodiments of the claimed subject mater and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be briefly mentioned or omitted so as to not unnecessarily obscure the embodiments of the claimed subject matter described. The examples used herein are intended merely to facilitate an understanding of ways in which the claimed subject matter may be practiced and to further enable those of skill in the art to practice the embodiments of the claimed subject matter described herein. Accordingly, the examples and embodiments herein are merely illustrative and should not be construed as limiting the scope of the claimed subject matter, which is defined solely by the appended claims and applicable law. Moreover, it is noted that like reference numerals represent similar parts throughout the several views of the drawings.

It is understood that the subject matter claimed is not limited to the particular methodology, protocols, devices, apparatus, materials, applications, etc., described herein, as these may vary. It is also to be understood that the terminology used herein is used for the purpose of describing particular embodiments only, and is not intended to limit the scope of the claimed subject matter.

Unless defined otherwise, all technical and scientific terms used herein have the same meanings as commonly understood by one of ordinary skill in the art.

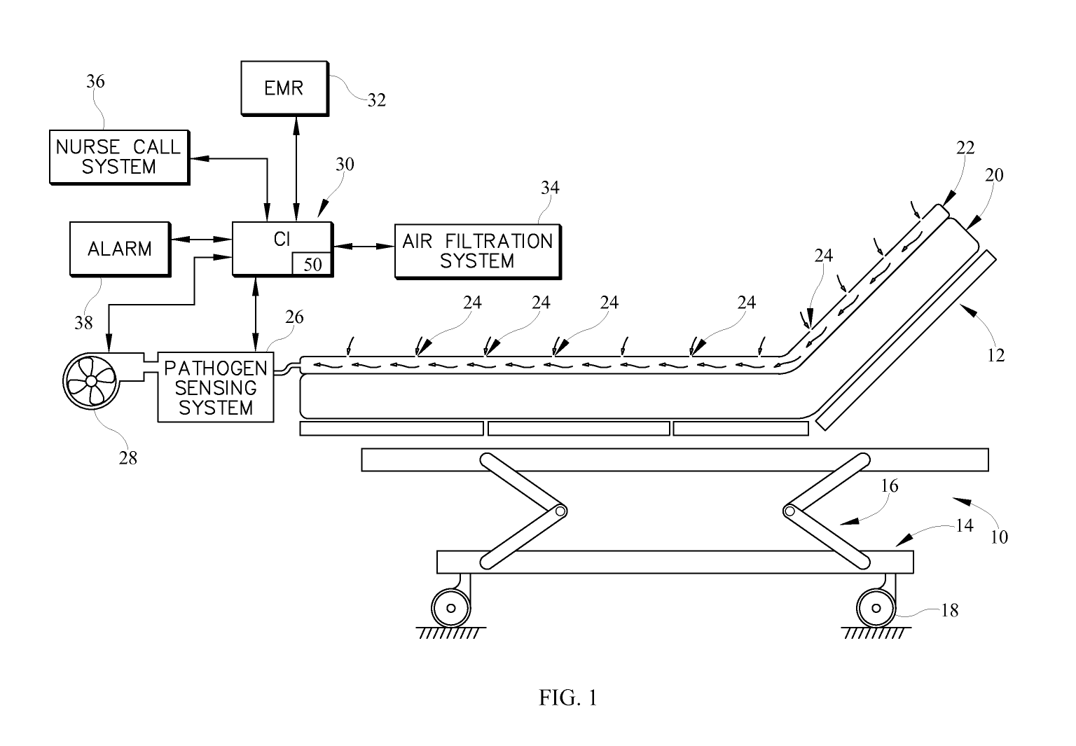

One embodiment of a system to detect a pathogen in the vicinity of a person support apparatus 10 is shown in FIG. 1. In this embodiment, the person support apparatus 10 is a bed, however, in other embodiments the person support apparatus 10 may be a wheelchair, stretcher or any other apparatus configured to support a person thereon. The person support apparatus 10 in this embodiment comprises an upper frame 12 which is supported over a lower frame 14 by supports 10. The upper frame 12 comprises one of more sections and the support 16 is configured to variably elevate at least one section of the upper frame 12 with respect to the lower frame 14. The lower frame 14 rests on at least one caster wheel 18 in this embodiment, allowing the person supported apparatus 10 to be transported. A person support surface 20 rests on the person support apparatus 10. In this embodiment the person support surface 20 is a mattress and comprises fluid filled bladders, in other embodiments the person support surface may be made of any combination of bladders, foam and other polymeric materials. A mattress topper 22 is configured to be positioned on top of the person support surface 20 such that a person can be supported on top of the mattress topper 22. The mattress topper 22 comprises fluid permeable pores 24 on its surface which is configured to support a person. Fluid permeable pores 24 are configured to allow air, water and microbial particles to pass through them. A pump 28 is configured to suck fluid in through the fluid permeable pores 24 and into a pathogen sensing system 26 through a fluidic connection as shown in FIG. 1. The pump 28 is configured to person one or more functions of the person support surface 20 including but not limited to inflation of bladders. In another embodiment the pump 28 is dedicated for use by the system to detect pathogens. In this embodiment the pathogen sensing system 26 comprises electrodes whose surfaces are polymerized. The constitution of the surroundings effect the electrical resistance measured between the electrodes and this change in resistance is used to identify the constitution of the surroundings by comparison with known resistance and/or change in resistance values. In other embodiments, any other type of sensors may be used including but not limited to metal oxide type semi-conductor (MOS) sensors and surface acoustic wave devices. In another embodiment, protein based biosensors such as odarant binding proteins (OBPs), sensory appendage proteins (SAPs), odorant or gustatory receptors, serpentine receptors and/or odarant degrading enzymes (ODEs) may be used along with electrochemical transducers to generate an electrical signal in response to detection of a chemical. The pump 28 and the pathogen sensing system 26 are configured to communicate with a caregiver interface (CI) 30. In this embodiment, the caregiver interface (CI) 30 comprises a memory 50 to store information supplied by the pathogen sensing system 26 for a predetermined time, a processor (not shown) to process information and a display (not shown). Memory 50 may be of any type including volatile and non-volatile. In this embodiment the CI 30 is mounted on the person support apparatus 10 and controls the function of one or more features of the person support apparatus 10. In another embodiment the system comprises a dedicated CI 30. The CI 30 is configured to communicate with a nurse call system 36 configured to alert a caregiver. The CI 30 in this embodiment is also configured to activate an alarm 30. Alarm 30 is an audio alert in this embodiment, in other embodiments alarm 30 may be any combination of audio, visual and/or tactile alerting system. The CI 50 is also configured to communicate with an electronic medical records (EMR) database 32. In this embodiment the CI 30 is configured to communicate with an air filtration system 34. The CI 30 is configured to control the air filtration system 34 based on the results provided by the pathogen sensing system 26. In one prophetic example if a high concentration of sweat is detected in the data transmitted by the pathogen sensing system 26 to the CI 30, the partial pressure of water vapor and/or temperature and/or volume of air being supplied to the room housing are modified to induce an environment to optimize sweat production. In another prophetic example, the air filtration system 34 comprises a controllable valve so as to prevent any air from leaking out of the room containing a patient without first cleansing the air if a determination is made that the data from the pathogen sensing system 26 indicates presence of a certain pathogen above a predetermined threshold. The air filtration system 34 may rely on any combination of ionization, electrostatic and mechanical filtration technologies.

During operation of the system shown in FIG. 1 the CI 30 is configured to control the operation of the pump 28 to create a suction pressure allowing suction of fluids through the fluid permeable pores 24 and into the pathogen sensing system 26. In this embodiment, the pathogen sensing system 26 communicates with the CI 30 and supplies to the CI 30 results of the testing on the fluid flowing into the pathogen sensing system 26. The CI 30 in this embodiment allows a caregiver to set thresholds for presence of one or more chemicals. The thresholds may be set in the form of limits or a rate of change. In another embodiment, the caregiver may program detection of specific combinations of chemicals as a threshold. These thresholds are stored in the memory 50 in this embodiment. When results obtained from the pathogen sensing system 26 indicate that a predetermined threshold has been exceeded the CI 30 activates alarm 38 in this embodiment. The CI 30 also communicates with a nurse call system 36 when the predetermined threshold has been exceeded. In this embodiment the CI 30 logs the results of testing provided by the pathogen sensing system 26 in an EMR 32 database, while in another embodiment the CI 30 obtains information from the EMR 32 which is used to determine the thresholds. The thresholds may be so selected that presence of any chemical or combinations of chemicals may be sought for detection. In this embodiment chemical signatures indicative of disease causing micro-organisms and/or vectors are sensed by the pathogen sensing system 26. In another embodiment results supplied by the pathogen sensing system 26 are evaluated by the graphical caregiver interface 30 to seek chemical signatures indicative of biochemical markers of wound development such as IL-1a and Creatine Phosphokinase (CPK).

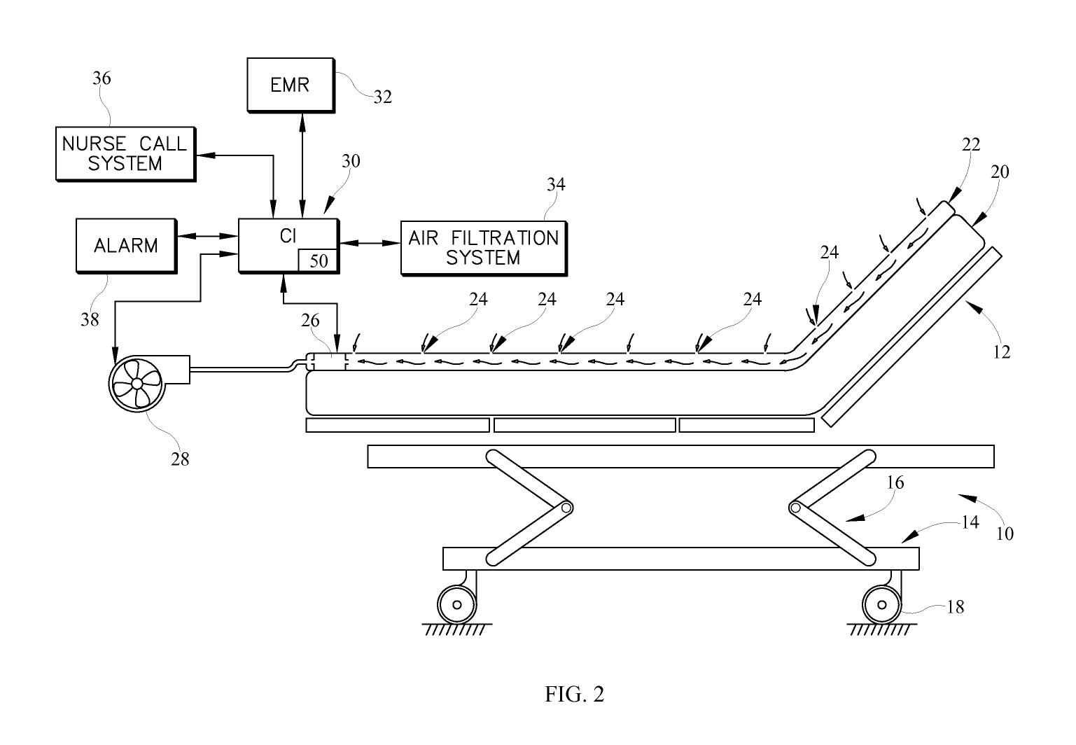

In the embodiment of a system to detect a pathogen in the vicinity of a person support apparatus 10 shown in FIG. 2, the pathogen sensing system 26 is housed within the mattress topper 22. In another embodiment the pump 22 is also housed within the mattress topper 22. The person support surface 20 in another embodiment comprises fluid permeable pores 24 and provides the fluidic path between the fluid permeable pores 24 and the pathogen sensing system 26.

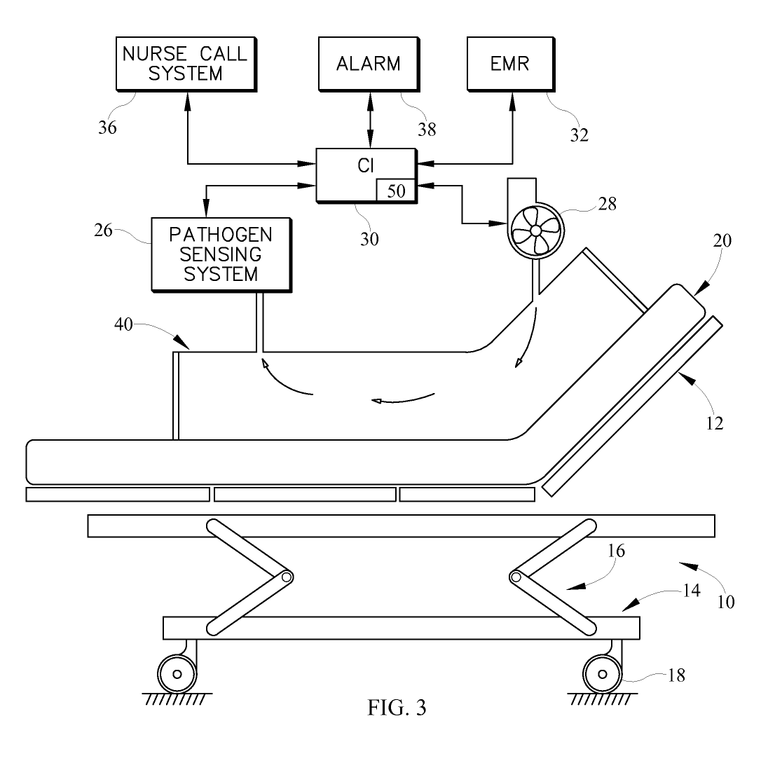

In FIG. 3 another embodiment of a system to detect a pathogen in the vicinity of a person is depicted. In the system shown in FIG. 3, a pathogen containment screen 40 is placed over the person support surface 20. The pathogen containment screen is made of a substantially fluid impermeable material and is configured to fit over at least a portion of a person supported by the person support apparatus 20. The pump 28 in controlled by CI 30 and supplies air with a positive pressure within the volume of air contained by the pathogen containment screen 40. The pathogen sensing system 26 is in fluidic communication with the volume of air contained by the pathogen containment screen 40. The pathogen sensing system 26 is configured to communicate with a CI 30 which comprises a memory 50. The CI 30 is also configured to communicate with a nurse call system 36, an alarm 38 and an EMR 32 in this embodiment.

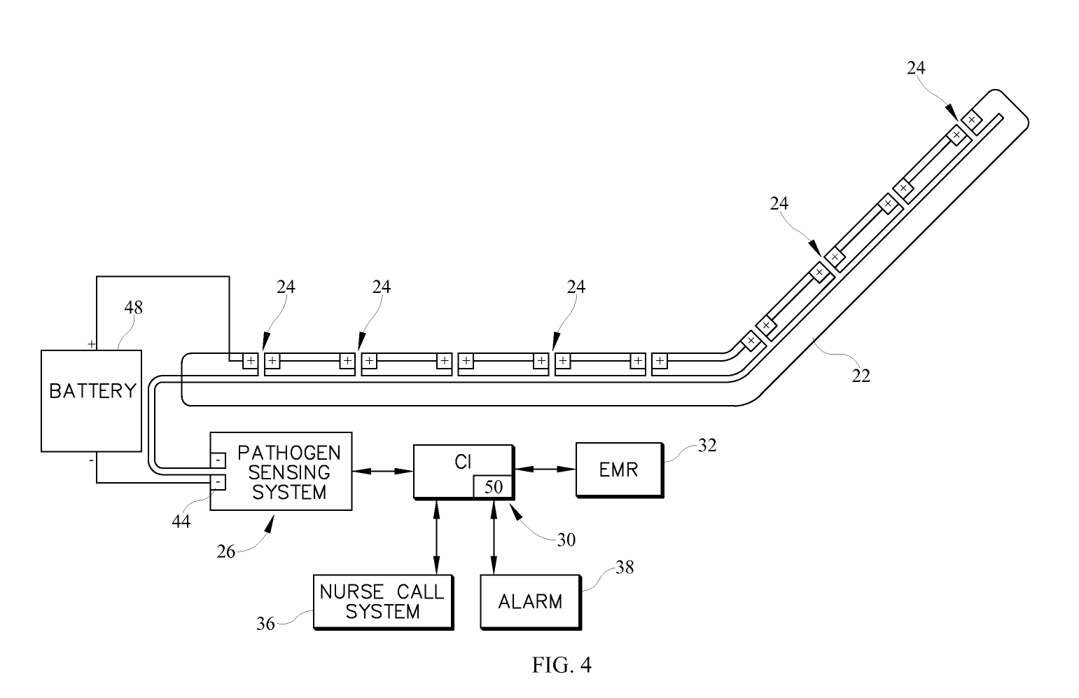

In the embodiment of a system to detect a pathogen in the vicinity of a person support apparatus 10 shown in FIG. 4 an electrical potential is used to induce flow of fluid between fluid permeable pores 24 and the pathogen sensing system 26. FIG. 4 shows a mattress topper 22 which comprises positively charged electrical leads 42 attached substantially close to the inlet of the fluid permeable pores 24 in this embodiment. Negatively charged electrical leads 44 are attached to the pathogen sensing system 26. The positively charged electrical leads 42 and negatively charged electrical leads 44 are attached to the positive and negative terminals of a battery 48 respectively. The battery 48 and electrical leads form a differential in electrical potential between the fluid permeable pores 24 and the pathogen sensing system 26. Particulate, volatile and liquid matters are drawn through the fluidic connection between the fluid permeable pores 24 and the pathogen sensing system 26 based on their electrophoretic mobility. In one embodiment the CI 30 provides electrical potential between the positively charged electrical leads 42 and the negatively charged electrical leads 44 instead of utilization of a battery 48. The pathogen sensing system 26 is in communication with a CI 30 comprising a memory 50. The CI 30 is configured to communicate with an EMR 32, nurse call system 36 and an alarm 38 in this embodiment. In another embodiment the fluid permeable pores 24, positively charged electrical leads 42 and the fluidic path to the pathogen sensing system 26 are incorporated in a person support surface 20.

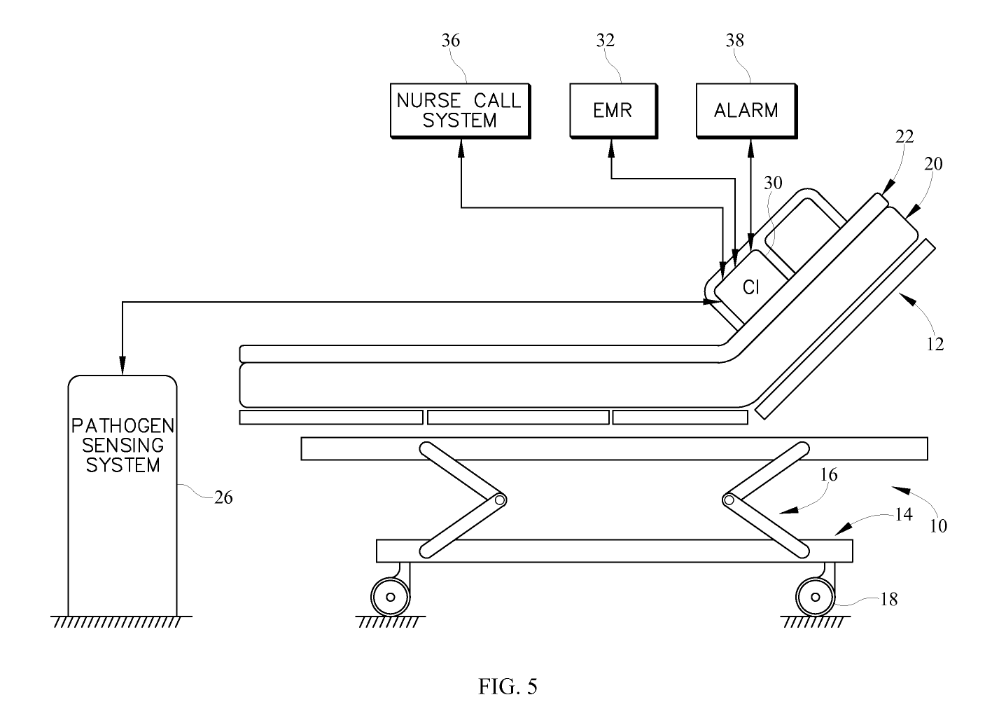

In the embodiment shown in FIG. 5, a standalone pathogen sensing system 26 samples air circulating around a person support apparatus and is configured to communicate the sensed data to a CI 30 of a person support apparatus 10. The stand alone pathogen sensing system 26 may be free standing structure resting on the floor, be mounted on the person support apparatus 10 or be supported by any other structure in the vicinity of the person support apparatus 10. CI 30 is configured to communicate with a nurse call system 36, EMR 32 and an alarm 38.

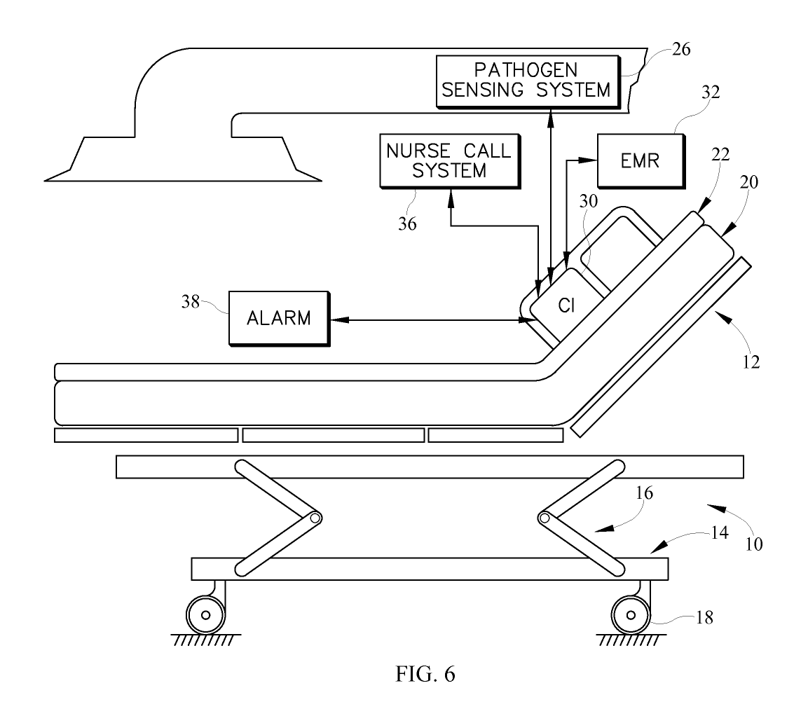

In another embodiment shown in FIG. 6, the pathogen sensing system 26 is housed within the air filtration system 34. Air filtration system 34 is the heating, ventilation and air conditioning (HVAC) system of the room which houses the person support apparatus 10. The pathogen sensing system 26 samples air flowing through the air filtration system 34 and is configured to communicate the sensed data to a CI 30 of a person support apparatus 10. The CI 30 is configured to control the air filtration system 34 based on the results provided by the pathogen sensing system 26. In one prophetic example if a high concentration of sweat is detected in the data transmitted by the pathogen sensing system 26 to the CI 30, the partial pressure of water vapor and/or temperature and/or volume of air being supplied to the room housing are modified to induce an environment to optimize sweat production. In another prophetic example, the air filtration system 34 comprises a controllable valve so as to prevent any air from leaking out of the room containing a patient without first cleansing the air if a determination is made that the data from the pathogen sensing system 26 indicates presence of a certain pathogen above a predetermined threshold. CI 30 is configured to communicate with a nurse call system 36, EMR 32 and an alarm 38.

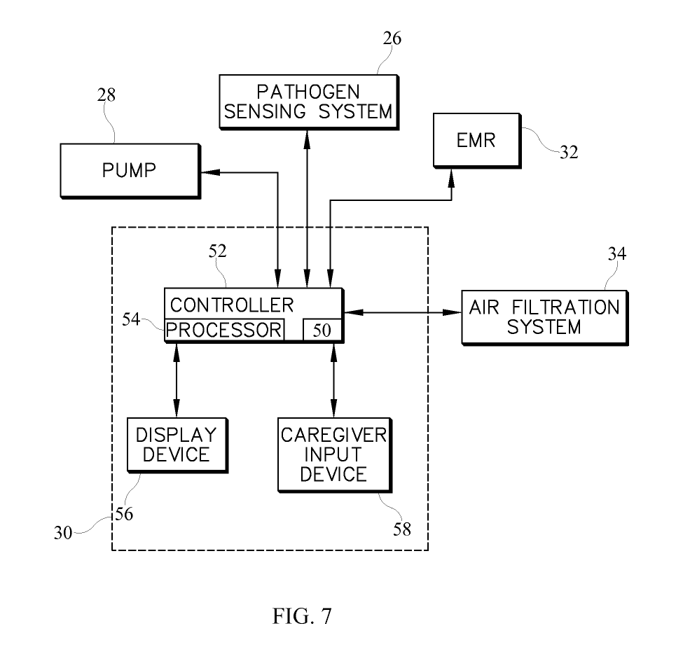

Another contemplated embodiment includes a control interface (CI) 30 including a controller 52 in communication with a display device 56 and a caregiver input device 58, the controller is physically different device from the display device 56 and the caregiver input device 58 in this embodiment as shown in FIG. 7. In this embodiment the controller 52 comprises a processor 54 and memory 50 in communication with a display device 56 and care giver input device 58. In this embodiment the display device 56 is a monitor and the care giver input device 58 is a pendant. In another embodiment, display device 56 and the caregiver input device 58 are portions of a touch sensitive screen.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

Preferred embodiments are described herein, including the best mode known to the inventor for carrying out the claimed subject matter. Of course, variations of those preferred embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

The disclosures of any references and publications cited above are expressly incorporated by reference in their entireties to the same extent as if each were incorporated by reference individually.

We claim:

1. A system for detecting pathogens for use with a person support apparatus, comprising:
a fluid transport system configured to transport fluid away from skin of a person supported by a person support apparatus;
at least one pathogen detection sensor, said at least one pathogen sensor configured to detect a pathogen in fluid transported in said fluid transport system;
a caregiver interface, said pathogen detection sensor configured to communicate with said caregiver interface which is configured to determine presence of a pathogen in a quantity exceeding a predetermined threshold, said caregiver interface configured to control at least one function of a person support apparatus.

2. The system of claim 1 wherein said fluid transport system comprises a pump to displace fluid.

3. The system of claim 2 wherein said pump is an electro-osmosis type pump.

4. The system of claim 2, wherein said pump uses positive pressure to transport fluid away from a person's skin.

5. The system of claim 1, wherein said at least one pathogen detection sensor is a metal oxide semi-conductor type sensor.

6. The system of claim 1, wherein said at least one pathogen detection sensor is an organic semi-conducting polymer type sensor.

7. The system of claim 1, wherein said at least one pathogen detection sensor is a surface acoustic wave type sensor.

8. The system of claim 1, wherein said at least one pathogen detection sensor is a protein based sensor.

9. The system of claim 1, wherein a portion of said fluid transport is integrated into a mattress topper.

10. The system of claim 1, wherein said caregiver interface is configured to communicate with an electronic medical record database.

11. The system of claim 1, wherein said caregiver interface is configured to communicate with a nurse call system.

12. The system of claim 1, wherein said caregiver interface is configured to communicate with an alarm.

13. The system of claim 1, wherein said caregiver interface is configured to communicate with an air filtration system.

14. The system of claim 1, wherein said caregiver interface comprises a controller.

15. The system of claim 1, wherein said caregiver interface comprises a display.

16. The system of claim 1 wherein said caregiver interface comprises a caregiver input device.

17. A system for detecting an odor in the vicinity of a hospital bed, comprising:
at least one odor sensor;
a fluid transport system configured to transport fluid away from skin of a person supported by a hospital bed to said at least one odor sensor;
a caregiver interface, said at least one odor sensor configured to communicate with said caregiver interface.

18. The system of claim 17 wherein said fluid transport system comprises a pump to displace fluid.

19. The system of claim 18 wherein said pump is an electro-osmosis type pump.

20. The system of claim 18, wherein said pump uses positive pressure to transport fluid away from a person's skin.

21. The system of claim 17, wherein said at least one odor sensor is a metal oxide semi-conductor type sensor.

22. The system of claim 17, wherein said at least one odor sensor is an organic semi-conducting polymer type sensor.

23. The system of claim 17, wherein said at least one odor sensor is a surface acoustic wave type sensor.

24. The system of claim 17, wherein said at least one odor sensor is a protein based sensor.

25. The system of claim 17, wherein a portion of said fluid transport system is integrated into a mattress topper.

26. The system of claim 17, wherein said caregiver interface is configured to communicate with an electronic medical record database.

27. The system of claim 17, wherein said caregiver interface is configured to communicate with a nurse call system.

28. The system of claim 17, wherein said caregiver interface is configured to communicate with an alarm.

29. The system of claim 17, wherein said caregiver interface comprises a controller.

30. The system of claim 17, wherein said caregiver interface comprises a display.

31. The system of claim 17, wherein said caregiver interface comprises a caregiver input device.

32. A system for purification of air in the vicinity of a person supported by a person support apparatus, comprising:
a person support apparatus;
a fluid transport system configured to transport fluid away from a person supported by the person support apparatus;
at least one chemical sensor configured to sense at least one chemical in fluid transported away from a person supported by the person support apparatus;
a caregiver interface, said at least one chemical sensor configured to communicate with said caregiver interface;
an air filtration system, said caregiver interface configured to communicate with said air filtration system.

33. The system of claim 32 wherein said a portion of said fluid transport system is integrated into a mattress topper.

34. A system for monitoring wound development, comprising:
a person support apparatus;
a fluid transport system configured to transport fluid away from a person supported by said person support apparatus;
at least one chemical sensor configured to sense at least one chemical in fluid transported away from a person supported by said person support apparatus;
a caregiver interface, said at least one chemical sensor configured to communicate with said caregiver interface, said caregiver interface comprising a memory configured to store signals supplied by the at least one chemical sensor for a predetermined time.

35. The system of claim 34 wherein said caregiver interface is configured to compare a signal supplied by the at least one sensor with an earlier signal supplied by said at least one chemical sensor and alert a caregiver if said comparison indicates a change greater than a predetermined threshold.

36. A method for detecting a chemical in the vicinity of a person support apparatus comprising:
transporting fluid away from a person supported by a person support apparatus;
testing said fluid transported from a person supported by said person support apparatus;
reporting result of said testing to a caregiver interface,
determining whether said result indicates presence of at least one chemical based on whether quantity of said at least one chemical detected is greater than a predetermined threshold;
activating an alarm to alert a caregiver when quantity of said at least one chemical detected is greater than a predetermined threshold.

37. The method of claim 36 further comprising the operation of determining if said at least one chemical is an indicator of presence of a pathogen.
